(19) United States
(12) Patent Application Publication
(10) Pub. No.: US 2024/0285327 A1
(43) Pub. Date: Aug. 29, 2024

(54) MANIFOLD FOR CRYOGENIC BALLOON CATHETER

(71) Applicant: Medtronic Ireland Manufacturing Unlimited Company, Dublin (IE)

(72) Inventor: Carlos H. Lima, Santa Rosa, CA (US)

(21) Appl. No.: 18/573,532

(22) PCT Filed: Jul. 19, 2022
(86) PCT No.: PCT/EP2022/070212

§ 371 Date: Dec. 22, 2023

Related U.S. Application Data
(60) Provisional application No. 63/226,922, filed on Jul. 29, 2021.

(51) Int. Cl. A61B 18/02; A61B 18/00

(52) U.S. Cl. CPC A61B 18/02; A61B 2018/00166; A61B 2018/0212

(57) ABSTRACT

A medical device includes an elongate shaft having an inner shaft is disposed within the elongate shaft, the inner shaft defines a first lumen defining a dedicated first port; an exhaust lumen offset from the first lumen and defining a dedicated second port; a second lumen offset from the first lumen and the exhaust lumen, the second lumen defining a dedicated third port; a third lumen offset from the first lumen, the exhaust lumen, and the second lumen, the third lumen defining a dedicated fourth port; an expandable member enclosing the dedicated first, second, third, and fourth ports. The dedicated first, second, third, and fourth ports are defined at different radial positions about the inner shaft.

### FIELD

The present technology is generally related to a manifold for a cryogenic balloon catheter.

### BACKGROUND

Cryotherapy is a useful treatment modality for many types of medical procedures. In some cases, it is desirable to administer cryotherapy from within a patient's body, such as from within a body lumen. Internal administration of cryotherapy can be advantageous, for example, in at least some neuromodulation procedures. These procedures can include percutaneously introducing a cryotherapeutic element into a patient and then advancing a catheter shaft carrying the cryotherapeutic element along an intravascular path to a suitable treatment location. Once positioned at the treatment location, the cryotherapeutic element can be cooled to modulate nearby nerves. The cooling caused by the cryotherapeutic element, for example, can reduce undesirable local or systemic sympathetic neural activity and thereby achieve various therapeutic benefits.

### SUMMARY

This disclosure generally relates to a manifold for a cryogenic balloon catheter.

A cryogenic balloon catheter can have a pressure monitoring tube, an inflow/capillary tube (which may be a thin polyimide tube), a thermocouple wire that travels along the length of shaft, and an exhaust lumen. Some aspects of the present disclosure involve the recognition that in cryogenic balloon catheters in which a pressure monitoring tube terminates at the location of an exhaust port, a negative pressure may be created that can result in incorrect pressure measurements when refrigerant is exhausted through the exhaust port. Some aspects of the present disclosure involve the recognition that kinking of a cryogenic balloon catheter may pose a risk to proper catheter operation by occluding the inflow of media delivering cold therapy.

In one aspect, the present disclosure provides a medical device, the medical device includes an elongate shaft. An inner shaft is disposed within the elongate shaft, the inner shaft defines a first lumen defining a dedicated first port; an exhaust lumen offset from the first lumen and defining a dedicated second port; a second lumen offset from the first lumen and the exhaust lumen, the second lumen defining a dedicated third port; a third lumen offset from the first lumen, the exhaust lumen, and the second lumen, the third lumen defining a dedicated fourth port; an expandable member enclosing the dedicated first, second, third, and fourth ports. The dedicated first, second, third, and fourth ports are defined at different radial positions about the inner shaft.

In another aspect of this embodiment, the device further includes an inflow tube affixed within the first lumen.

In another aspect of this embodiment, the first lumen is closed at a position distal to the dedicated first port and proximal to a distal end of the inner shaft.

In another aspect of this embodiment, the exhaust lumen is closed at a position distal to the dedicated second port and proximal to a distal end of the inner shaft.

In another aspect of this embodiment, the device further includes a thermocouple wire affixed within the second lumen.

In another aspect of this embodiment, the second lumen is closed at a position distal to the dedicated third port and proximal to a distal end of the inner shaft.

In another aspect of this embodiment, the device further includes a pressure monitoring tube affixed within the third lumen.

In another aspect of this embodiment, the third lumen is closed at a position distal to the dedicated fourth port and proximal to a distal end of the inner shaft.

In another aspect of this embodiment, the inflow tube extends outward from the dedicated first port.

In another aspect of this embodiment, the dedicated first, second, third, and fourth ports are defined at different longitudinal positions about the inner shaft.

In one aspect, a medical device includes an elongate shaft having an inner shaft disposed within the elongate shaft. The inner shaft defines a first lumen sized defining a dedicated first port; an exhaust lumen offset and fluidly isolated from the first lumen and defining a dedicated second port; a second lumen offset and fluidly isolated from the first lumen and the exhaust lumen, the second lumen being sized to receive a thermocouple wire, the second lumen defining a dedicated third port; a third lumen offset and fluidly isolated from the first lumen, the exhaust lumen, and the second lumen, the third lumen being sized to receive a pressure monitoring tube and defining a dedicated fourth port. An expandable member encloses the dedicated first, second, third, and fourth ports.

In another aspect of this embodiment, the device further includes an inflow tube affixed within the first lumen.

In another aspect of this embodiment, wherein the first lumen is closed at a position distal to the dedicated first port and proximal to a distal end of the inner shaft.

In another aspect of this embodiment, the exhaust lumen is closed at a position distal to the dedicated second port and proximal to a distal end of the inner shaft.

In another aspect of this embodiment, the device, further includes the thermocouple wire affixed within the second lumen.

In another aspect of this embodiment, the second lumen is closed at a position distal to the dedicated third port and proximal to a distal end of the inner shaft.

In another aspect of this embodiment, the device further includes the pressure monitoring tube affixed within the third lumen.

In another aspect of this embodiment, the third lumen is closed at a position distal to the dedicated fourth port and proximal to a distal end of the inner shaft.

In another aspect of this embodiment, the dedicated first, second, third, and fourth ports are defined at different radial positions about the inner shaft.

In one aspect, a medical device includes an elongate shaft having an inner shaft disposed within the elongate shaft. The inner shaft defines a first lumen sized to receive an inflow tube and defining a dedicated first port; an exhaust lumen offset and fluidly isolated from the first lumen and defining a dedicated second port; a second lumen offset and fluidly isolated from the first lumen and the exhaust lumen, the second lumen being sized to receive a thermocouple wire, the second lumen defining a dedicated third port; a third lumen offset and fluidly isolated from the first lumen, the exhaust lumen, and the second lumen, the third lumen being sized to receive a pressure monitoring tube and defining a dedicated fourth port. The inflow tube is affixed within the first lumen. The thermocouple wire is affixed within the second lumen. The pressure monitoring tube is affixed within the third lumen. An expandable member encloses the dedicated first, second, third, and fourth ports and distal ends of the inflow tube, thermocouple wire, and pressure monitoring tube.

Further disclosed herein is a medical device that includes an elongate shaft having an inner shaft is disposed within the elongate shaft, the inner shaft defines a first lumen defining a dedicated first port; an exhaust lumen offset from the first lumen and defining a dedicated second port; a second lumen offset from the first lumen and the exhaust lumen, the second lumen defining a dedicated third port; a third lumen offset from the first lumen, the exhaust lumen, and the second lumen, the third lumen defining a dedicated fourth port; an expandable member enclosing the dedicated first, second, third, and fourth ports, wherein the dedicated first, second, third, and fourth ports are defined at different radial positions about the inner shaft.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a system view of an exemplary cryogenic medical system and medical device constructed in accordance with the principles of the present application;

FIG. 2 is a side inside view of the distal end of the medical device shown in FIG. 1 with the expandable member removed;

FIG. 3 is a perspective view of the distal end shown in FIG. 2;

FIG. 4 is a perspective view of a proximal end of the inner shaft of the medical device shown in FIG. 1 sliced to show structures within; and

FIG. 5 is a perspective view of another embodiment of the distal end of the medical device shown in FIG. 1 with the expandable member removed.

### DETAILED DESCRIPTION

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

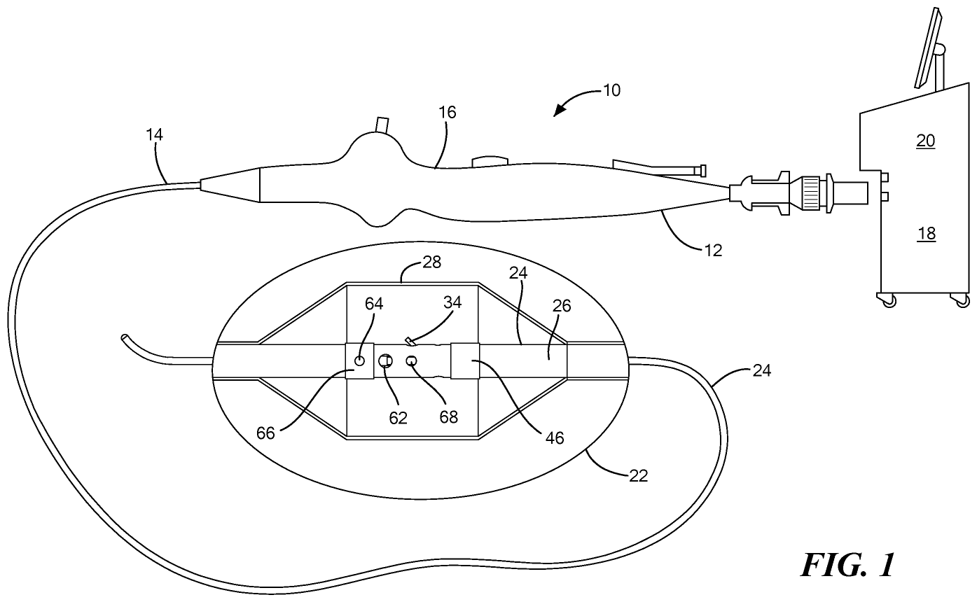

Referring now to the drawings in which like reference designators refer to like elements, there is shown in FIG. 1 an exemplary medical system constructed in accordance with the principles of the present application and designated generally as “10.” The system may include a medical device 12 including a proximal end 14 in fluid communication with a handle 16, which is further in fluid communication with a console 18 having a refrigerant source 20. The console 18 is configured to deliver refrigerant, such as nitrous oxide, to the medical device 12.

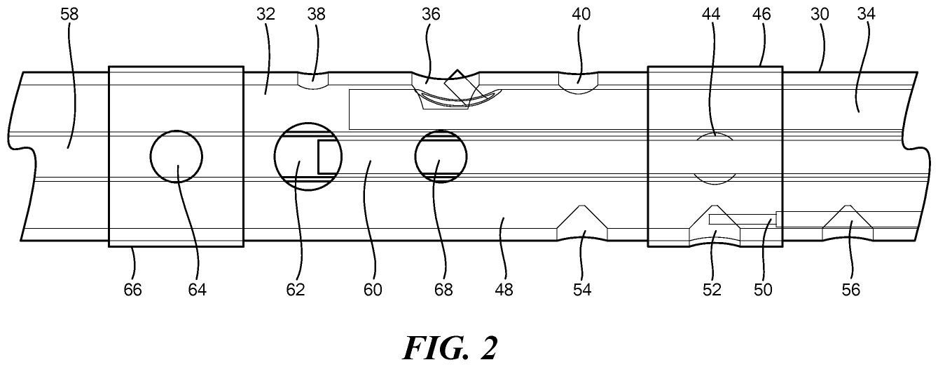

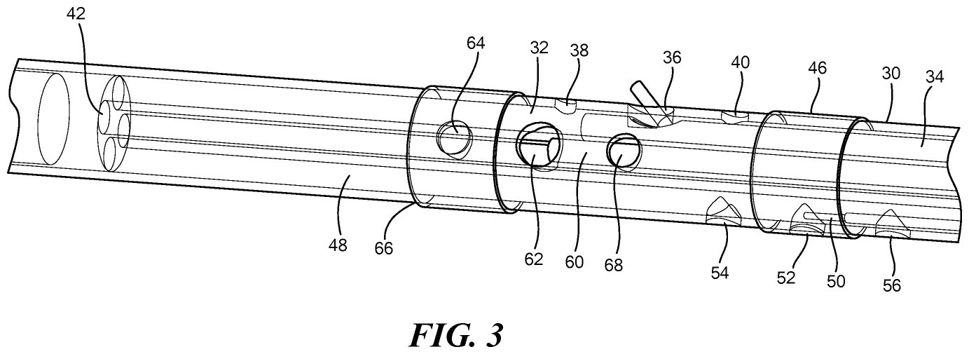

Referring now to FIGS. 1-4, the medical device 12 may further include a distal end 22 about which cryogenic energy is exchanged between the medical device 12 and a target tissue, for example, renal tissue (e.g., for renal denervation). For example, the medical device 12 may include an elongate shaft 24 disposed between the proximal end 14 and the distal end 22 and which further defines a lumen 26 there through. An expandable element 28 is disposed proximate the distal end 22 about the elongate shaft 24 and in fluid communication with the lumen 26. The expandable member 28 may be, for example, an inflatable balloon configured to exchange thermal energy with tissue. An inner shaft 30 is deposed within the lumen 26 and within a portion of the expandable element 28. For example, the inner shaft 30 may extend through the expandable member 28. The inner shaft 30, which may be braided for reinforcement, may define a first lumen 32 sized to receive an inflow tube 34 and further defines a dedicated first port 36. As used herein, the term “dedicated,” referenced with any of the ports described herein, means a port that provides fluid access solely to the structure in which it is defined. For example, the dedicated first port 36 facilitates the inflow tube 34 infusing cryogenic fluid into the expandable member 28. In one configuration, the inflow tube 34 extends at least partially outward from the dedicated first port 36. In one configuration, the first lumen 32 may be closed at a position distal to the dedicated first port 36. For example, as shown in FIG. 2, an aperture 38 may be drilled into the inner shaft 30 and bond filled to prevent flow from the inflow tube 34 from flowing distal to the dedicated first port 36. Moreover, an aperture 40 proximal to the dedicate first port 36 may also be bond filled to secure to the inflow tube 34 to the inner shaft 30 to prevent movement of the inflow tube 34.

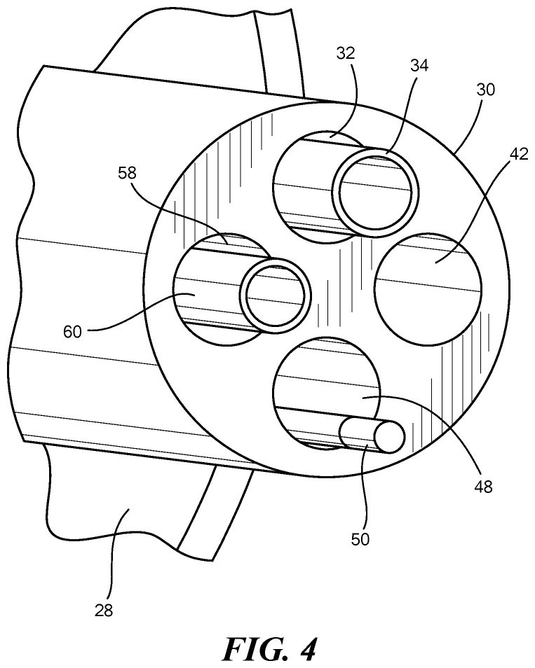

Continuing to refer to FIGS. 1-4, an exhaust lumen 42 (shown in FIG. 4) is offset from the first lumen 32 within the inner shaft 30 and defines a dedicated second port 44. In one configuration, the exhaust lumen 42 is radially offset from the first lumen 36 at for example a 90-degree angle. The dedicated second port 44 may be defined at a position proximal to the dedicated first port 36 may further be defined in a first radiopaque marker band 46. The exhaust lumen 42 is configured to be in communication with a vacuum source (not shown) to extract cryogenic fluid from the expandable member 28.

Continuing to refer to FIGS. 1-4, a second lumen 48 is offset from the first lumen 32 and the exhaust lumen 42. In one configuration, the second lumen 48 may be disposed at an angle of 180 degrees from the first lumen 32 and 90 degrees from the exhaust lumen 42. The second lumen 48 is sized to receive a thermocouple wire 50 configured to measure a temperature within the expandable member 28. The second lumen 48 defines a dedicated third port 52 in which a distal end of the thermocouple wire 50 is adjacent to for fluid access to the expandable member 28. Similar to the first lumen 32, an aperture 54 may be distal to the dedicated third port 52 and be bond filled to close the second lumen 48 and an aperture 56 may be proximal to the dedicated third port 52 and may also be bond filled to secure the thermocouple wire 50 to the second lumen 48.

A third lumen 58 may be included and offset from the first lumen 32, the exhaust lumen 42, and the second lumen 48. For example, in one configuration, the third lumen 58 may be radially offset from the first, second and the exhaust lumens. In one configuration the third lumen 58 is disposed and 90 degrees from the second lumen 48 and 90 degrees from the first lumen 32. The third lumen 58 is sized to receive a pressure monitoring tube 60 configured to measure a pressure with the expandable member 28 and further defines a dedicated fourth port 62. In an exemplary configuration, the dedicated fourth port 62 is distal to dedicated first port 36, dedicated second port 44, and dedicated third port 52. Similar to the other first lumen 32 and second lumen 48, the third lumen 58 defines an aperture 64 distal to the dedicated fourth port 62 which is bond filled to closed to third lumen 58. In one configuration, the aperture 64 is defined in a second radiopaque marker band 66 distal to the dedicated fourth port 66. Moreover, an aperture 68 proximal to the dedicated fourth port 62 may be included and bond filled to secure the pressure monitoring tube 60 within the third lumen 58. In one configuration, the expandable member 28 encloses the dedicated first, second, third, and fourth ports. Moreover, the dedicated first, second, third and fourth ports may each be radially disposed about the inner shaft 30 at an angle of 90 degrees with respect to each other. That is, each dedicated port may be disposed on a different face of the inner shaft 30.

Isolating a lumen from all other lumens, such as, for example, shown and described in this disclosure, may prevent or reduce incidence of tangling and/or kinking. Isolation may improve ease of manufacture, such as, for example, by reducing or avoiding the drilling of holes into the inner shaft 34. Some or all lumens can be isolated in this manner. Moreover, by isolating the respective lumens in which the thermocouple wire 50 and the pressure monitoring tube 60 reside from the other lumens within the inner shaft 34, interference with the accuracy of temperature and pressure measurements, respectively, by fluids exiting, entering or within other lumens can be reduced or avoided. Additionally or alternatively, isolation of lumens may prevent or reduce the occurrence of occlusion of respective lumens.

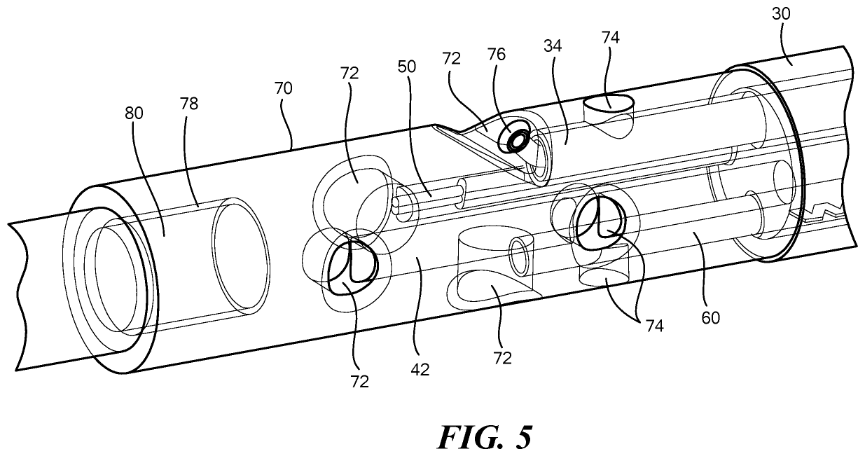

Referring now to FIG. 5, in another embodiment of the distal end 22 of the medical device 12, the inner shaft 30 may be co-terminus with the elongate shaft 24 proximate the expandable member 28. In particular, rather than extending through the expandable member 28, the inner braided shaft 30 terminates within a proximal portion of the expandable member 28. The pressure monitoring tube 60, the exhaust lumen 42, the inflow tube 34, and the thermocouple wire 50 all extend away from the distal end of the inner shaft 30 into a rigid hub 70. The rigid hub 70 may be radiopaque and may be, for example, a metal or metal alloy. In one configuration, the rigid hub 70 defines the same outer diameter to that of the inner shaft 30, but in other configurations, may define an outer diameter less than or greater than the outer diameter of the inner shaft 30. In this configuration, it is not necessary to drill holes in any marker bands or the inner shaft 30, which may reduce bowing or buckling of the device 12 proximate the expandable member 28.

As with the embodiments described above, each of the pressure monitoring tube 60, the exhaust lumen 42, the inflow tube 34, and the thermocouple wire 50 includes a dedicated port 72 and is also bonded to the rigid hub 70 through corresponding bonding ports 74 at positions proximal to the corresponding dedicated port 72. For example, each bonding port may be prefabricated such that a bonding agent may be injected therein to bond the corresponding structure to the hub 70. In one configuration, the inflow tube 34 includes a capillary tube 76 extending from the inflow tube 34 at an oblique angle and the corresponding port 72 is cut at an oblique angle with respect to the rigid hub 70. The rigid hub 70 may further define a recess 78 sized to receive a soft tip 80 configured to extend distally away from the expandable member 28.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

Further disclosed herein is the subject-matter of the following clauses:

1. A medical device, comprising:
an elongate shaft;
an inner shaft disposed within the elongate shaft, the inner shaft defining:
   a first lumen defining a dedicated first port;
   an exhaust lumen offset from the first lumen and defining a dedicated second port;
   a second lumen offset from the first lumen and the exhaust lumen, the second lumen defining a dedicated third port;
   a third lumen offset from the first lumen, the exhaust lumen, and the second lumen, the third lumen defining a dedicated fourth port;
an expandable member enclosing the dedicated first, second, third, and fourth ports; and
wherein the dedicated first, second, third, and fourth ports are defined at different radial positions
   about the inner shaft.

2. The device of clause 1, further including an inflow tube affixed within the first lumen.

3. The device of clause 2, wherein the first lumen is closed at a position distal to the dedicated first port and proximal to a distal end of the inner shaft.

4. The device of clause 1 or of any of the preceding clauses, wherein the exhaust lumen is closed at a position distal to the dedicated second port and proximal to a distal end of the inner shaft.

5. The device of clause 1 or of any of the preceding clauses, further including a thermocouple wire affixed within the second lumen.

6. The device of clause 5, wherein the second lumen is closed at a position distal to the dedicated third port and proximal to a distal end of the inner shaft.

7. The device of clause 1 or of any of the preceding clauses, further including a pressure monitoring tube affixed within the third lumen.

8. The device of clause 7, wherein the third lumen is closed at a position distal to the dedicated fourth port and proximal to a distal end of the inner shaft.

9. The device of clause 1 or of any of the preceding clauses, wherein the inflow tube extends outward from the dedicated first port.

10. The device of clause 1 or of any of the preceding clauses, wherein the dedicated first, second, third, and fourth ports are defined at different longitudinal positions about the inner shaft.

11. A medical device, comprising:
an elongate shaft;
an inner shaft disposed within the elongate shaft, the inner shaft defining:
   a first lumen sized defining a dedicated first port;
   an exhaust lumen offset and fluidly isolated from the first lumen and defining a dedicated second port;
   a second lumen offset and fluidly isolated from the first lumen and the exhaust lumen, the second lumen being sized to receive a thermocouple wire, the second lumen defining a dedicated third port;
   a third lumen offset and fluidly isolated from the first lumen, the exhaust lumen, and the second lumen, the third lumen being sized to receive a pressure monitoring tube and defining a dedicated fourth port; and an expandable member enclosing the dedicated first, second, third, and fourth ports.

12. The device of clause 11, further including an inflow tube affixed within the first lumen.

13. The device of clause 12, wherein the first lumen is closed at a position distal to the dedicated first port and proximal to a distal end of the inner shaft.

14. The device of clause 11 or of any of clauses 11-13, wherein the exhaust lumen is closed at a position distal to the dedicated second port and proximal to a distal end of the inner shaft.

15. The device of clause 11 or of any of clauses 11-14, further including the thermocouple wire affixed within the second lumen.

16. The device of clause 15, wherein the second lumen is closed at a position distal to the dedicated third port and proximal to a distal end of the inner shaft.

17. The device of clause 11 or of any of clauses 11-16, further including the pressure monitoring tube affixed within the third lumen.

18. The device of clause 17, wherein the third lumen is closed at a position distal to the dedicated fourth port and proximal to a distal end of the inner shaft.

19. The device of clause 11 or of any of clauses 11-18, wherein the dedicated first, second, third, and fourth ports are defined at different radial positions about the inner shaft.

20. A medical device, comprising:
an elongate shaft;
an inner shaft disposed within the elongate shaft, the inner shaft defining:
   a first lumen sized to receive an inflow tube and defining a dedicated first port;
   an exhaust lumen offset and fluidly isolated from the first lumen and defining a dedicated second port;
   a second lumen offset and fluidly isolated from the first lumen and the exhaust lumen, the second lumen being sized to receive a thermocouple wire, the second lumen defining a dedicated third port;
   a third lumen offset and fluidly isolated from the first lumen, the exhaust lumen, and the second lumen, the third lumen being sized to receive a pressure monitoring tube and defining a dedicated fourth port;
the inflow tube affixed within the first lumen;
the thermocouple wire affixed within the second lumen;
the pressure monitoring tube affixed within the third lumen; and
an expandable member enclosing the dedicated first, second, third, and fourth ports and distal ends of the inflow tube, thermocouple wire, and pressure monitoring tube.

21. A medical device, comprising:
an elongate shaft;
an inner shaft disposed within the elongate shaft, the inner shaft including a rigid hub extending from a distal end of the inner shaft, the hub defining:
   a first lumen defining a dedicated first port;
   an exhaust lumen offset from the first lumen and defining a dedicated second port;
   a second lumen offset from the first lumen and the exhaust lumen, the second lumen defining a dedicated third port;
   a third lumen offset from the first lumen, the exhaust lumen, and the second lumen, the third lumen defining a dedicated fourth port;
an expandable member enclosing the dedicated first, second, third, and fourth ports; and
wherein the dedicated first, second, third, and fourth ports are defined at different radial positions about the inner shaft.

1. A medical device, comprising:
an elongate shaft;
an inner shaft disposed within the elongate shaft, the inner shaft defining:
a first lumen defining a dedicated first port;
an exhaust lumen offset from the first lumen and defining a dedicated second port;
a second lumen offset from the first lumen and the exhaust lumen, the second lumen defining a dedicated third port;
a third lumen offset from the first lumen, the exhaust lumen, and the second lumen, the third lumen defining a dedicated fourth port;
an expandable member enclosing the dedicated first, second, third, and fourth ports; and
wherein the dedicated first, second, third, and fourth ports are defined at different radial positions about the inner shaft.

2. The device of claim 1, further including an inflow tube affixed within the first lumen.

3. The device of claim 2, wherein the first lumen is closed at a position distal to the dedicated first port and proximal to a distal end of the inner shaft.

4. The device of claim 1, wherein the exhaust lumen is closed at a position distal to the dedicated second port and proximal to a distal end of the inner shaft.

5. The device of claim 1, further including a thermocouple wire affixed within the second lumen.

6. The device of claim 5, wherein the second lumen is closed at a position distal to the dedicated third port and proximal to a distal end of the inner shaft.

7. The device of claim 1, further including a pressure monitoring tube affixed within the third lumen.

8. The device of claim 7, wherein the third lumen is closed at a position distal to the dedicated fourth port and proximal to a distal end of the inner shaft.

9. The device of claim 1, wherein the inflow tube extends outward from the dedicated first port.

10. The device of claim 1, wherein the dedicated first, second, third, and fourth ports are defined at different longitudinal positions about the inner shaft.

11. A medical device, comprising:
an elongate shaft;
an inner shaft disposed within the elongate shaft, the inner shaft defining:
a first lumen sized defining a dedicated first port;
an exhaust lumen offset and fluidly isolated from the first lumen and defining a dedicated second port;
a second lumen offset and fluidly isolated from the first lumen and the exhaust lumen, the second lumen being sized to receive a thermocouple wire, the second lumen defining a dedicated third port;
a third lumen offset and fluidly isolated from the first lumen, the exhaust lumen, and the second lumen, the third lumen being sized to receive a pressure monitoring tube and defining a dedicated fourth port; and
an expandable member enclosing the dedicated first, second, third, and fourth ports.

12. The device of claim 11, further including an inflow tube affixed within the first lumen.

13. The device of claim 12, wherein the first lumen is closed at a position distal to the dedicated first port and proximal to a distal end of the inner shaft.

14. The device of claim 11, wherein the exhaust lumen is closed at a position distal to the dedicated second port and proximal to a distal end of the inner shaft.

15. The device of claim 11, further including the thermocouple wire affixed within the second lumen, wherein optionally the second lumen is closed at a position distal to the dedicated third port and proximal to a distal end of the inner shaft.

16. The device of claim 11, further including the pressure monitoring tube affixed within the third lumen, wherein the optionally third lumen is closed at a position distal to the dedicated fourth port and proximal to a distal end of the inner shaft.

17. The device of claim 11, wherein the dedicated first, second, third, and fourth ports are defined at different radial positions about the inner shaft.

18. A medical device, comprising:
an elongate shaft;
an inner shaft disposed within the elongate shaft, the inner shaft defining:
a first lumen sized to receive an inflow tube and defining a dedicated first port;
an exhaust lumen offset and fluidly isolated from the first lumen and defining a dedicated second port;
a second lumen offset and fluidly isolated from the first lumen and the exhaust lumen, the second lumen being sized to receive a thermocouple wire, the second lumen defining a dedicated third port;
a third lumen offset and fluidly isolated from the first lumen, the exhaust lumen, and the second lumen, the third lumen being sized to receive a pressure monitoring tube and defining a dedicated fourth port;
the inflow tube affixed within the first lumen;
the thermocouple wire affixed within the second lumen;
the pressure monitoring tube affixed within the third lumen; and
an expandable member enclosing the dedicated first, second, third, and fourth ports and distal ends of the inflow tube, thermocouple wire, and pressure monitoring tube.

19. A medical device, comprising:
an elongate shaft;
an inner shaft disposed within the elongate shaft, the inner shaft including a rigid hub extending from a distal end of the inner shaft, the hub defining:
a first lumen defining a dedicated first port;
an exhaust lumen offset from the first lumen and defining a dedicated second port;
a second lumen offset from the first lumen and the exhaust lumen, the second lumen defining a dedicated third port;
a third lumen offset from the first lumen, the exhaust lumen, and the second lumen, the third lumen defining a dedicated fourth port;
an expandable member enclosing the dedicated first, second, third, and fourth ports; and
wherein the dedicated first, second, third, and fourth ports are defined at different radial positions about the inner shaft.
